(19) United States
(12) Patent Application Publication
(10) Pub. No.: US 2024/0307610 A1
(43) Pub. Date: Sep. 19, 2024

(54) SINGLE-USE MEDICAL ITEM HAVING AN INTEGRATED MARKER

(71) Applicant: B. Braun Melsungen AG, Melsungen (DE)

(72) Inventors: Christoph Erlen, Kassel (DE); Andreas Katerkamp, Melsungen (DE); Markus Discher, Knuellwald (DE); Maria Buerger, Malsfeld (DE); Jan Schwarz, Melsungen (DE)

(21) Appl. No.: 18/576,472

(22) PCT Filed: May 10, 2022
(86) PCT No.: PCT/EP2022/062667

§ 371 Date: Jan. 4, 2024

(30) Foreign Application Priority Data
Jul. 5, 2021 (DE) 10 2021 117 339.6

(51) Int. Cl. A61M 5/14

(52) U.S. Cl. CPC A61M 5/14; A61M 2205/273; A61M 2205/6054; A61M 2205/6081

(57) ABSTRACT

A single-use medical item can be used with a medical device, a medical system, a medical method and a computer-readable storage medium. The single-use medical item, which can be a medical tube or a medical sliding clamp, includes at least one integrated marker as an identifying feature. The marker is a predefined portion of the single-use item. The predefined portion has a color, magnetization, a geometric shape, such as indentation patterns, a predefined conductivity, and/or an RFID tag embedded in the single-use item.

### CROSS-REFERENCE TO RELATED APPLICATIONS

This application is the United States national stage entry of International Application No. PCT/EP2022/062667, filed on May 10, 2022, and claims priority to German Application No. 10 2021 117 339.6, filed on Jul. 5, 2021. The contents of International Application No. PCT/EP2022/062667 and German Application No. 10 2021 117 339.6 are incorporated by reference herein in their entireties.

### FIELD

The present disclosure relates to a single-use medical item/accessory item adapted to be inserted in or to be connected to a medical device. In addition, the present disclosure relates to a set of single-use medical items (single-use item set), a medical device, in particular an infusion pump, to or in which a single-use item can be connected or inserted, a medical system comprising a single-use item set and a medical device, a medical method, and a computer-readable storage medium.

### BACKGROUND

Medical devices, such as, for instance, infusion pumps, require single-use medical items and/or accessory items for an operation and a therapy of a patient, which items are inserted in this device prior to the beginning of the treatment. From a plurality of insertable single-use items, particular single-use items may, as required and adapted to the patient, be selected, inserted into the medical device and be used appropriately. The type or kind of single-use item inserted by the user is usually related to the therapy required or to the patient IV line used. For instance, a plurality of different kinds of single-use infusion items may be inserted in a volumetric infusion pump. In addition to a drug administration this may also be the administration of enteral nutrition solutions through the stomach, which are guided to the patient by means of a single-use infusion item.

Currently, a user/operator, here medical professionals such as, for instance, a nurse or a doctor, selects manually on a graphical user interface (GUI) first of all a single-use medical item and then inserts it manually in the medical device. This, however, takes place without the user input being checked. Moreover, current medical devices have the disadvantages that the manual selection of both a drug and a single-use infusion item requires high effort and always a high concentration of the user.

If, for instance, the user inadvertently selects a critical or even life-threatening combination of a drug and a single-use item for the patient, the medical devices are not capable of prohibiting such selection and such operation. Specific handling of potentially unsafe or critical combinations of single-use infusion items and drugs (or other data) selected from the drug library is not provided for in the operating procedure of the infusion pump.

Neither is there any possibility of linking the administration of a drug or the conducting of a therapy with a special prerequisite that a selected, particular single-use infusion item is used. For instance, the use of a single-use item with UV protection should be provided if the drug transported through the single-use item is UV-sensitive or light-sensitive.

The state of the art therefore completely lacks an intelligent relation between single-use items, on the one hand, and drugs with corresponding drug data, on the other hand, which may i.e. comprise drug-specific standard settings and boundaries for the infusion parameters.

### SUMMARY

It is therefore an object of the present disclosure to avoid or to at least mitigate the disadvantages from the state of the art and to provide in particular a single-use medical item, a set of single-use medical items (single-use item set), a medical device, a medical system, a use, a medical method, and a computer-readable storage medium which improve the treatment and the safety of a patient and increase operating ergonomics for a user. A further partial object consists in ensuring, on the basis of an inserted or introduced single-use item, a limitation of access to drugs and/or non-compatible settings so as to reliably avoid mistakes. Also, a further partial object may in particular be not to admit an unsafe combination of single-use item(s) and drug(s) so as to prevent possible application errors.

A basic principle of the present disclosure thus consists in that a single-use medical item/accessory item, such as an infusion tube, is configured such that it comprises at least one predefined, integrated marker as an identifying feature. When this single-use item is then inserted (i.e. received or connected) in a medical device, the identifying feature is detected by an appropriate detection system of the medical device. From the detected identifying feature the medical device may draw then conclusions about the single-use item, in particular its identification (for instance, a class or type or ID of the single-use item) and/or a position of the single-use item (for instance, so as to check whether it has been connected correctly). Specifically, on the basis of the detected identifying feature an adaptation of a drug selection and/or a selection of a further (suitable) single-use item may then take place.

In other words, a technical principle for identification of the single-use item (accessory element), in particular a single-use infusion item, and a resulting adaption of the medical device behaviour/appliance behaviour is thus described in the present disclosure, in particular by means of a filtering of a drug library and/or a single-use item data set. Thus, an intelligent examination and adaption so to say takes place, which increases safety of a patient's treatment. Specifically, in accordance with the present disclosure a filtering of the drug library can be performed on the basis of the single-use item selection or the detected single-use item.

Specifically, ergonomics of operation, such as, for instance, a manipulation of the drug library in the user interface (UI), of the medical device, in particular the infusion pump, may be improved this way. Furthermore, a limitation of the access to drugs and other settings, in particular in the drug library, can be performed on the basis of the single-use item inserted. Also, unsafe combinations of single-use items and drugs may be prohibited for preventing application errors. Another advantage consists in that the single-use items and single-use item sets as well as the medical device and systems enable new concepts by the possibility of linking therapy forms of the pump to the use of specific single-use items (e.g. a specific single-use item set for PCA therapy).

An identification and/or a mechanism for identification of the single-use item, in particular single-use infusion item, enables the ergonomic use of the drug library of the medical device, in particular on the infusion pump, by the use of filters and/or filter mechanisms. Specifically, unsafe combinations of single-use infusion items and drugs are prevented.

Advantageous embodiments will specifically be explained in the following.

### Single-Use Item

The object of the present disclosure is solved in that the single-use item comprises an integrated marker as an identifying feature.

In other words, in accordance with the present disclosure a single-use medical item, in particular a medical tube or a medical sliding clamp, is provided for use in a medical device, in particular an infusion pump, which is characterised in that the single-use medical item comprises at least one integrated marker as an identifying feature, wherein the at least one marker is a predefined portion of the single-use item having a (specific) color (from a predefined, discrete amount of colors which each have a characteristic assigned to) and/or a predefined portion of the single-use item having (permanent) magnetisation or a material with a magnetic effect and/or a predefined portion of the single-use item having a predefined geometric shape, in particular in the form of indentation patterns, and/or a portion of the single-use item having a predefined conductivity and/or an embedded RFID tag.

The expression integrated marker here means an inherent identifying feature imprinted on the single-use item.

In accordance with one embodiment the single-use item may comprise a portion with a magnetised structure, in particular a magnetised or magnetisable plastic, as an identifying feature. In this manner, an (identifying) information may be integrated as a magnetic information in the single-use item. It is not visible and may especially be used for a branding, i.e. a company designation, so as to make the single-use item tamper-proof and to characterise it as an original part. Likewise, the magnetic identifying feature may also provide information for an identification. Specifically, during manufacturing magnetisable or magnetised particles may be admixed to a plastic which is used as a material in the portion of the single-use item, preferably the entire single-use item. The magnetisation may provide a new identifying feature for a corresponding detection by a medical device and in particular an appropriate filtering. Furthermore, the magnetisation may preferably be used for a specific orientation of the single-use item since, for instance, a specific spatial position and/or orientation of the magnetisation are imprinted on the single-use item for orientation. Since a (permanent) magnetic field comprises a static orientation, this orientation may be used for the position information. Also, an individual magnet may be inserted in the single-use item, which forms the marker and provides the magnetic identifying feature. In other words, a possible variant of the identifying feature is thus a magnetic information, in particular as a magnetised plastic, wherein preferably different magnetisations (orientations and/or strengths) for different single-use items such as different tubes or different sliding clamps. In one embodiment a magnet is inserted in the sliding clamp. The magnetic information of the identifying feature may be used to detect the presence of the single-use item, in particular a sliding clamp, or to identify and appropriately judge a (relative) position of the single-use item, in particular the sliding clamp, due to different magnetisations. A “different magnetisation” means in particular a different strength of magnetisation and/or a different spatial position of the magnetisation and/or a different strength of the magnetisation at different portions of the single-use item. Preferably, a magnet may be inserted in the single-use item so as to perform detection of a presence of a connection and/or a position of the connection.

In accordance with a further embodiment the identifying feature may be predefined portion of the single-use item having a special geometric shape or a geometric pattern. The inserted geometric pattern is designed in particular in the form of indentations/grooves/recesses with a specific mutual arrangement. Specifically, a correct orientation of the single-use item may be ensured by means of the geometry and further information for a detection may be inserted. For instance, at a circumference of an infusion tube, indentations/grooves extending in an axial direction may be inserted so as to insert a marking as an original product and to aggravate imitation. Furthermore, the geometric shapes may be used for implementing an identification of the single-use item. A characteristic of the identifying feature may thus be a predefined geometry, in particular in the form of indentations/grooves/recesses in hose clamps as single-use items.

In accordance with a further embodiment a predefined portion of the single-use item may be provided with a color, for instance, red, blue, yellow, or green, and enable an identification of the single-use item by means of the visible color. Thus, for instance, classes of specific single-use items, such as, for instance, tubes may be characterised, e.g. infusion tubes for UV-sensitive drugs with the color blue so as to enable an identification of the tubes and/or tube classes by means of the color (by means of an assignment table). A color sensor, for instance, a camera with subsequent color identification, may then determine the class by means of the color and may, for instance, provide it to a drug library for a filtering. A possible characteristic of the identifying feature is thus a color. Specifically, infrared markers and/or ultraviolet markers are inserted in the single-use item. IR markers and UV markers are not visible to the eye and are only detected by the excitation of a particular wavelength range or by an appropriate sensor. Specifically, individual particles may be inserted in the single-use item as UV marker and/or IR marker in the material. The single-use item has specific absorption/reflection/or transmission spectres.

Preferably, the single-use item may comprise a predefined portion of the single-use item having a predefined conductivity. The portion of an infusion tube which is usually inserted in an infusion pump may, for instance, be configured in the form of a cylindrical sheathing as a coating or as a cylindrical tube portion. By the choice of a suitable material for at least a particular region of a surface of the single-use item an electric conductivity can be set on the basis of the material. Specifically, this conductivity σ is minimally 1×10\hat{ }2 S/m, preferably 1×10\hat{ }3 S/m, and/or maximally 60×10\hat{ }6 S/m, preferably 1×10\hat{ }5 S/m. This characteristic is not visible to the human eye, so that an invisible branding can be provided (exactly one conductivity), on the one hand, and, on the other hand, especially by different surface materials of different conductivity at a specific partial face of the single-use item, for instance, an outer jacket of a tube end, may be provided for an identification feature.

Specifically, the single-use item may comprise an RFID tag as an integrated marker. This RFID tag is inseparably connected to the single-use item and serves, for instance, for identification and transmission of information. Moreover, position identification may be performed, and a specific filtering may be performed on the basis of the position identification of the singe-use item. A variant may therefore comprise a radio module or a transponder, such as, for instance, an RFID tag. It may especially be used for position identification and/or for identifying the single-use item.

Specifically, the single-use item may be a tube, in particular an infusion tube, or a (medical) sliding clamp which is adapted to be fastened to a tube and to clamp same. Specifically, the single-use item may be a tube with a sliding clamp. In the case of a sliding clamp as a single-use item in accordance with the present disclosure, tubes may be manufactured uniformly and be marked subsequently only, which is of advantage for production and the manufacturing costs.

Preferably, the single-use item may comprise a combination of a first identifying feature and a second identifying feature. In other words, the single-use item may comprise at least two integrated markers of different kind. An infusion tube as a single-use item may, for instance, comprise a magnetic information (magnetic marker) as a first identifying feature and a color (color marker) as a second identifying feature. Specifically, it is possible to orient the infusion tube on the basis of the magnetic structure, so that, for instance, the portion with the color is arranged toward a color sensor, and subsequently an identification of the infusion tube takes place by means of the color. Alternatively or additionally a magnetic structure may be inserted in a sliding clamp, so that the sliding clamp can be oriented in the clamp module so as to orient the measurement position at the sliding clamp optimally relative to the color sensor. Alternatively, a branding may be examined by means of the magnetisation, and only in the case of successful examination a color identification may be performed in a next step. By a specific combination of various identifying features an orientation and/or identification can take place even better. Specifically, the first identifying feature (of the first marker) my cause a first limitation of a drug selection (limit drug basis), and based thereon the second identifying feature may achieve a further limitation of the drug selection. For instance, a type of the single-use item, for instance, a tube for a parenteral nutrition or a UV-proof tube, may be determined by means of the first identifying feature, and an even more exact limitation, for instance, a nutrition solution for chemotherapy patients or a UV-proof tube for an infusion solution, may be performed by means of the second identifying feature. An individual limitation for a dedicated customer may take place. In even other words, the first feature (e.g. a color) may be used for a first coarse filtering, such as, for instance, a parenteral nutrition solution, and the second feature (e.g. an RFID tag) may be provided for a filtering to the specific customer or the specific clinic. The first feature may thus be used, for instance, for limiting the drug basis (by means of the first feature), and a further limitation takes place, for instance, by the color as a second identifying feature. Specifically, the identifying features may be variable, for instance, by a software/application, for instance, an RFID tag as an identifying feature, so that individual limitations for a dedicated customer or a particular clinic may be generated.

Specifically, the single-use item may comprise a portion with a predefined magnetisation and a portion with a predefined conductivity. By means of the magnetisation it is possible to first detect the presence of the single-use item and to subsequently perform a conductivity test to perform an identification of the single-use item.

In accordance with one embodiment the single-use item may comprise at least two integrated markers of different kind, wherein a first marker as a first identifying feature is a predefined portion with a magnetisation and/or a geometric shape and/or a predefined conductivity, and a second marker as a second identifying feature is a predefined portion with a color so as to have a combined or two-stage identification performed by a medical device. Preferably, two different functions, namely a presence identification and a color identification, may thus be combined by two different features. Especially the magnetisation and the conductivity are not visible to the user from outside. In other words, especially by magnetisations or also specific conductivities the presence, for instance, of a sliding clamp in a clamp module of a medical device can be identified. Only once it has been identified positively will the color identification be performed in a next step. Apart from a product branding another function, the identification of a presence of a sliding clamp in the clamp module, is thus usable. A kind of magnetisation, in particular an orientation and/or a strength, additionally allows conclusions about the position of the sliding clamp in the clamp module. This reveals whether the sliding clamp has been inserted wrongly or correctly,

Preferably, the single-use item may comprise a combination of the integrated marker as an IR marker and a magnetic marker.

In accordance with a further embodiment the single-use item may comprise at least two markers of different kind, wherein a first marker as a first identifying feature is a radio transponder, in particular an RFID tag, and a second marker as a second identifying feature is a predefined portion with a color. Specifically, a check code may be stored in the RFID tag, which is embedded in an encoded manner with a private key only by the manufacturer and can be verified by means of a public key. Thus, checking for an original part may take place and only subsequently (if positive) a color identification may be performed. Specifically, an RFID tag as a marker is integrated on a tube or a tube clamp. Alternatively or additionally a drug basis may also be selected by means of the read-out information of the RFID tag and a specific number of drugs or only one drug may be selectable by means of the color. Thus, a color and a radio module and/or a transponder may be used as markers and hence as identifying features.

In accordance with one embodiment the single-use item may comprise a portion with a predefined geometry and a portion with a specific color. The geometry may in particular be configured as at least two spaced-apart bores or grooves of the single-use item, in particular a tube clamp. Specifically, a holding contour may be provided as a geometry for a clear orientation, and in the case of a correct orientation the color identification may take place. Alternatively or additionally the geometry may again be provided for branding and authenticity verification. By inserting indentations/a geometric marking on a sliding clamp, a product branding (for instance, marking as a B.Braun product) may be achieved, for instance. The color identification and the geometry information, especially in the form of bores, grooves or other geometries of the tube clamp, are used to enable the color identification by means of the branding only when the product branding has been checked successfully. The predefined geometry of a sliding clamp may be used to enable a holding contour for orienting the sliding clamp in the clamping module.

In accordance with a further embodiment the first identifying feature may be a conductivity and the second identifying feature may be a color. A product branding may be inserted in the sliding clamp as a single-use item in that a specific predefined electric conductivity is introduced in the material. The color identification is enabled and performed when the conductivity has been checked successfully.

Preferably, the single-use item may be an infusion tube and the infusion tube may comprise, on at least one end portion at its radially outer surface, a color coating or an insertion of color pigments into its basic structure (into its basic material), which differs from its basic color, especially white-transparent, and preferably magnetised particles/magnetic particles may further be introduced as markers in the end portion of the infusion tube so as to provide a magnetisation as a further, second identifying feature.

Preferably, the single-use item may comprise an infrared marker (IR marker) and/or an ultraviolet marker (UV marker) as a color marker. An IR marker and a UV marker are not in a range visible to the human eye and are consequently not identifiable without additional means. If these markers are excited by a light source with an appropriate wavelength, an excited radiation emitted by the markers can be detected. In this manner, it is possible to insert a marking for providing an identifying feature, on the one hand, and to achieve a kind of branding of the single-use item. By means of an examination by a detection system it is then possible to confirm authenticity of the single-use item. Specifically, a specific pattern of UV and/or IR markers may also be inserted in the single-use item, for instance, in the form of a QR code or an annulus or a ring. If the single-use item is an infusion tube, circumferential rings which are arranged along a longitudinal axis at a distance from one another may be formed preferably at one end of the infusion tube by means of a UV marker. For instance, two rings arranged concentrically to the longitudinal axis of the infusion tube 20) may be provided by UV markers at the end of the tube which is plugged to a connection of a medical device. A detection system of the medical device which is adapted to UV markers detects the two rings as an identifying feature of the tube and determines, for instance, that it is the matter of an original product of the manufacturer, and that the two rings are suited for an infusion administration of a drug. Thus, it may be encoded (invisibly) in the infusion tube that it is an original product, which further increases a patient's safety, and it may additionally be an identification of the infusion tube (by means of an appropriate assignment table, for instance “two rings”-“suited for drug administration”) as a single-use item suited for drug administration. Since the markers are inserted directly in the single-use item and are inseparably connected therewith, for instance, in that the UV markers or IR markers are moulded into the material to be formed directly 30) during the manufacturing of a sliding clamp or a tube, security against forgery can be established and an identification with corresponding information can be achieved.

Specifically, the various identifying features may hence be combined in a similar manner. Thus, a color marker as an identifying feature may be used as a “branding” and the color identification and the underlying reduction of the drug selection in the drug library may likewise be implemented.

Specifically, the single-use medical item may be an infusion tube or a sliding clamp of an infusion tube/an infusion line, or a clip at a beginning or an end of a pump segment of an infusion tube, or a cassette surrounding the infusion tube, or a part of the channel carrying liquid, or an air filter or a particle filter, or a syringe, or a single-use infusion item, or an attachment part/attachment module which is firmly connected with the single-use item, preferably is adapted to be coupled and decoupled. The identifying feature is each provided on this single-use item and/or the marker is placed as an identifying feature. Specifically, the marker may be provided as an identifying feature on a cylinder and/or on a piston of a syringe. Specifically, the single-use item may be a single-use infusion item and may be formed as an infusion tube with or without a pump segment (e.g. similar to Space Line), preferably with a cassette. Alternatively, the single-use item may be a syringe. In one embodiment a single-use infusion item may also be treated and reused.

Possible characteristics of the at least one identifying feature on the single-use item, in particular the single-use infusion item, may be: at least one of the following identifying features or combinations of two or several identifying features thereof: colors and/or specific absorption/reflection/or transmission spectres, in particular with a portion of a relevant spectrum in the infrared range and/or a portion of the relevant spectrum in the UV range; computer-readable 2D codes, in particular bar codes or QR codes; holograms; magnetic information; predefined geometries with which a kind of signature is inserted in the single-use item; a radio (incl. RFID); and/or a conductivity.

Specifically, additional characteristics of the identifying feature may be that it is designed such that, on its basis, the user (by means of his/her sensory organs) and also a medical device with an appropriate detection system, in particular an infusion pump, is capable of identifying which single-use item and hence which form of therapy is concerned. Specifically, this may be performed by the color of a tube clamp on the infusion tube, which may further also have a more general signalling effect, such as, for instance, red color=“attention” or “critical drug”, or purple color=“enteral nutrition”.

### Single-Use Item Set

Furthermore, the object of the present disclosure with respect to a single-use medical item set comprising at least one first single-use medical item and one second single-use medical item is solved in that the first single-use item and the second single-use-item are each especially a single-use item in accordance with the present embodiment and comprise a first marker as a first identifying feature and a second marker as a second identifying feature, wherein the first marker of the first single-use item is of a configuration different from that of the second marker of the second single-use item so as to differentiate the first single-use item from the second single-use item. Preferably, the first single-use item and the second single-use item is thus a single-use item in accordance with the present disclosure, and the identifying feature of the first single-use item is of a configuration different from that of the second single-use item so as to enable an identification.

In accordance with one embodiment the first and second single-use items may each comprise a portion having a magnetisation as a marker, wherein a spatial position/orientation of the magnetisations is different and/or a strength of a magnetisation, in particular at different portions of the single-use item, is different so as to differentiate the two single-use items by means of the magnetisation. Due to the different characteristic of the magnetisation the first single-use item may be encoded quasi invisibly relative to the second single-use item. Specifically, a magnetisation of the first single-use item may be different from a magnetisation of the second single-use item, and especially a spatial position of the magnetisation may be different and/or a strength of a magnetisation at different portions of the single-use item may be different.

In accordance with a further embodiment the first single-use item may be a first infusion tube and the second single-use item may be a second infusion tube, wherein an end portion of the first infusion tube comprises a first color at its radially outer surface, and an end portion of the second infusion tube comprises a second color at its radially outer surface, which is different from the first color, and wherein preferably furthermore the end portion of the first and second infusion tubes comprises a magnetisation as a further identifying feature, which is, however, of equal configuration in the first and second infusion tubes so as to enable an identification and/or orientation of the first and second infusion tubes.

Preferably the single use item set may, as the at least first and second single-use items, comprise an identical item, in particular a sliding clamp, which each comprise a (first) magnetic marker which is of identical configuration with both single-use items so as to ensure an original part examination and to enable an identification of an orientation of the single-use item, and comprise a second marker as an identifying feature, which is in the first single-use item of a configuration different from that of the second single-use item.

In accordance with one embodiment the single-use item set may also comprise two different types of single-use items, for instance, an infusion bag and an infusion tube, which each comprise a color portion of identical color as a marker, so as to indicate that these two single-use items belong together, wherein an RFID tag with different encoding is additionally inserted in each single-use item so as to enable a differentiation and identification.

### Medical Device and Medical System

The objects are fulfilled with respect to a medical device, in particular an infusion pump, in that it comprises:
a receptacle or a connection for at least one single-use item, in particular for a single-use infusion item;
a detection system, in particular with at least one sensor, preferably with a pump sensor, for detecting at least one integrated marker adapted in particular to the detection system, as an identifying feature of the single-use item inserted or connected; and
a control unit provided and adapted to identify the single-use item by means of the provided detected identifying feature and/or to determine a position of the single-use item relative to the detection system.

In accordance with one embodiment the medical device may further comprise:
a storage unit with a drug library in which data concerning drugs are stored;
preferably a single-use item library stored in the storage unit, in which data for at least two different single-use items are stored; and
a control unit which is provided and adapted, by means of the detected and hence identified single-use item:
   to filter and/or to limit a drug selection possibility in the in drug library
   and/or to filter and/or to limit a selection possibility of a further single-use item
   and/or when setting a therapy of the medical device with a predefined combination of drug or drug concentration and single-use item to output an alarm and/or to prohibit a start of the therapy.

In accordance with a further embodiment the detection system may comprise a magnetic sensor configured to detect a magnetic field of the single-use item as an identifying feature, and comprises further a color sensor configured to detect a color of a predefined portion of the received or connected single-use item, wherein the control unit is configured, in a first stage, to determine a correct position and hence a correct reception or a correct connection of the single-use item to the medical device by means of the detected magnetic field and, in the case of a correct position of the single-use item, to perform an identification of the single-use item on the basis of the color detected by the color sensor in a second stage.

Preferably, the medical device may further comprise a position determining module, in particular a GPS module or a communications module, preferably a WLAN module, with a stored assignment of communications network to position determination, and the control unit may be configured to filter and/or to limit a drug selection possibility in the drug library and/or to filter and/or to limit a selection possibility of a single-use item by means of the determined position of the medical device. In other words, the medical device may preferably comprise a communications module, in particular a WLAN module, for position determination for a first filtering. If, for instance, in the case of an infusion pump the communications module determines that it is used in an oncological ward, the control unit filters, on the basis of this position, a selection of single-use items usable and/or a drug administration (which drug and in which concentration/dose). Specifically, the control unit may be provided with an assignment table of IP addresses with therapy wards with appropriate therapies from a storage unit, for instance, the IP address 192.168.10.11 for the oncological ward for an oncological therapy. By means of a color as an identifying feature and a color sensor of the medical device it is then possible to perform a second filtering. In even other words, the position determination which is implemented by means of a communications module in the infusion pump (e.g. via WLAN) may be used to limit the drug selection of the infusion pump starting out from the position of the pump (a priori knowledge about the ward). A further limitation of the drug selection is furthermore performed by the identified single-use item (by means of its color/by means of color identification).

Specifically, the medical device comprises an electric conductivity sensor adapted to determine an electric conductivity of the single-use item at a predefined portion. For instance, in a connection for the single-use item two electric contact points may be provided which, in the case of a connected single-use item, are in direct contact therewith and which enable an appropriate measurement of the electric conductivity of the connection of the single-use item.

In one embodiment the medical device may comprise an RFID reader for reading out a connected or inserted single-use item with an RFID tag as a marker.

In a further embodiment the medical device may comprise an (exchangeable) connection adapter comprising a geometric counter structure adapted to a connection geometry of a single-use item (identifying feature), so that only a single-use item with a matching structure can be connected.

The objects are fulfilled with respect to a medical system in that it comprises:

a medical device, in particular a medical device in accordance with the present disclosure, in which a single-use item/accessory item may be received or to which a single-use item can be connected; and at least one insertable single-use item, in particular in accordance with the present disclosure, comprising at least one integrated marker as an identifying feature which can be detected by a detection system of the medical device which is adapted to the marker. Specifically, the medical system comprises a single-use item set comprising at least one first and second single-use item, in particular a single-use item set in accordance with the present disclosure, which each comprise at least one predefined (mutually different) identifying feature which is detected by the detection system of the medical device so as to detect and differentiate the two single-use items.

In accordance with a preferred embodiment the first and second single-use items may each comprise an RFID tag as an integrated marker for an identifying feature, and the medical device may comprise an RFID reader which digitally provides the read-out data of the RFID tag to the control unit, and the control unit is adapted to limit, on the basis of a patient library with data stored for patients, a drug selection possibility in the drug library and to indicate the selection possibility to a user by means of a display, in particular an OP monitor.

A medical device may in particular be an infusion pump with a corresponding combination with single-use items/accessory items. Also, the medical device may be a dialysis machine with specific dialysis items as single-use items/accessory items, in particular dialysis sets as single-use item sets, in accordance with the present disclosure, wherein the identification of the identifying features of the dialysis item and/or the dialysis set is used for filtering the possible therapy options and/or device settings. Furthermore, a medical device may be configured in the form of a lung ventilator which comprises specific (ventilation) tube sets as single-use sets, and wherein the identification of at least one identifying feature is used to automatically perform filtering of the possible therapy options and/or device settings.

Specifically, the medical device may comprise a light sensor, and a clamp orientation (geometry) relative to the light sensor and/or angle to the detection system may be provided as an identifying feature.

Specifically, a medical device, a medical system, and a medical method are disclosed which have the following concept:

The medical device may in particular be configured as an infusion pump, particularly preferred as a tube pump or syringe pump. The medical device comprises a detection system with at least one sensor which is adapted to detect an identifying feature of a single-use item. Specifically, this sensor may be a pump sensor. Furthermore, the medical device comprises a storage unit with a drug library in which entries concerning various drugs and preferably concerning therapies with these drugs are stored. Preferably, a single-use item library with entries with a list of the available single-use items (single-use item data set) may also be provided in the storage unit so as to establish a linking and/or combination with the drug library for the drugs and the single-use library for the single-use items. Furthermore, the medical device comprises a control unit. Preferably, the medical device further has a display, in particular a touch display for input. In other words, an (infusion) pump as a medical device may enable the user to program the infusion by means of an input device and a display and to monitor the administration thereof. In this process, a control unit of the infusion pump controls the administration of the infusion. Specifically, the control unit provides the necessary data to the input device and the display. The control unit then processes the inputs made by the user by means of the input device. When inserting or placing the single-use item, in particular the single-use infusion item, or in an inserted state at a later point of time during a treatment, the single-use (infusion) item is identified by the medical device, in particular the infusion pump, by means of its identifying feature by the detection system.

As a single-use item, in particular a single-use infusion item may be used which comprises a liquid guidance for an infusion medium, for instance, a bag and/or a tube. Examples of infusion media are, for instance, solutions with drugs or solutions with electrolytes or nutrition solutions. The single-use item comprises a marker as an identifying feature which is configured to match/to be adapted to the detection system and/or to at least one sensor of the detection system. The possibility of differentiation of a number of different single-use infusion item types on the basis of the identifying feature and/or their combination is important. Specifically, the marker and/or the identifying feature is adapted to the sensor, in particular pump sensor, of the detection system.

The control unit is specifically adapted to use the single-use item identification of the single-use item for any of the following actions (as purposes) or a combination thereof:

The control unit may indicate a filtering of the drug selection in the drug library on the display (a kind of comfort feature). Filter possibilities include, for instance, a limitation of the selectable drugs on the basis of the identified single-use infusion item so as to prevent, for instance, that the wrong drug is administered to the wrong patient IV line. A further filter possibility is a limitation of the selectable therapy and an infusion setting on the basis of the identified single-use (infusion) item. For instance, a PCA therapy can be conducted with a specific single-use infusion item only. Alternatively or additionally an action may be that the control unit informs the user by means of an output device such as a display about potentially critical constellations during the starting of the therapy and in particular prevents the activation of such therapy (a safety feature). These possible critical constellations may, for instance, be that the drug selected by the user in the drug library or its concentration do not match with an identified type of the single-use item. For instance, the critical constellation may exist that a nutrition solution is used enterally with a single-use item for an IV line. Another critical constellation may, for instance, exist if an administration of an unsuited drug is to be performed in a peridural space. Specifically, filtering of the single-use item selection on the display may take place during the insertion procedure (a comfort feature) to give direct feedback to the user. The subsequently selected single-use item is then used in the control unit for the activating or setting of parameters which are relevant for the operation of the single-use infusion item inserted. Specifically, the medical device then detects this selected and inserted single-use item and thus checks the correct implementation. The parameters of the medical device to be set on the basis of the detected single-use item may be inter alia: A parameter for controlling delivery (e.g. a delivery amount or a delivery course) and/or a parameter for controlling a pressure detection and/or a parameter for controlling an air detection and/or a parameter for defining a maximal dwell time of the single-use item, in particular with a corresponding visual alert after expiration of the dwell time, and/or a parameter concerning an air-stop-filter (yes/no). Specifically, the parameters to be activated or to be set may be stored in the single-use item data set so as to achieve a linking of a single-use item to the parameter to be set.

The filter methods in the control unit may comprise a mechanism taking the selection automatically without any further user interaction if the filter result on the basis of the identifying feature comprises one hit only. Specifically, the drug library may comprise per drug (or another structural element) information/data which may be used for the identifying feature-dependent filtering. Alternatively or additionally the single-use item data set may comprise information per single-use item (and/or data may be stored in a storage unit) which may be used for an identifying feature-dependent filtering.

Specifically, identification on the medical device side, in particular the infusion pump, may be performed by a (pump) sensor of the detection system or an interaction between the (pump) sensor and the control unit. An evaluation of the at least one identifying feature takes place statically (detection of a color, does not change with movement and not with time, either) or dynamically/transiently (changes with time, especially conditioned by a movement).

In accordance with a further embodiment, the medical device may comprise a detection system and a control device and may be adapted to perform a “dynamic identification” of a single-use item, especially to determine a hysteresis curve of a single-use item to identify a single-use item. Specifically, a medical system with a medical device and a single-use item set with a first and a second single-use item, each of which comprises a magnetised structure, may comprise a magnetic sensor which detects a hysteresis curve during a dynamic inserting or a connection of the single-use item and then performs the appropriate filtering and/or limitation on the basis of the hysteresis curve.

One kind of transfer of the identifying feature may, for instance, be that the single-use item always emits itself, for instance, generates a magnetic field, or the single-use item is actively excited by the medical device, in particular the infusion pump. For instance, the medical device may also emit an electromagnetic radiation which excites a radiation of particles in the material, which are then emitted correspondingly (excited radiation with corresponding specific absorption/reflection/or transmission spectres). Or an RFID tag is excited with an associated frequency and then emits electromagnetic waves with appropriate information which are collected by the medical device. Alternatively, the single-use item may also be excited by energy from the surroundings/environment.

Specifically, the medical device, in particular the infusion pump, may also use further filter criteria additionally. The following attributes may be used individually or in combination: a position of the use; a current or planned time of use; a pump type; a serial number of the pump; information concerning the patient treated (i.e. size, gender, body surface, age, kind of disease, other assignment to a patient population), therapy proposals provided via the communications network; and/or a treating health facility or organisation unit in the health facility.

### Method and Computer-Readable Storage Medium

Specifically, a method for the optimised operation of infusion pumps by means of detection of the single-use item is disclosed. With respect to a medical method for the optimised operation of a medical device, in particular an infusion pump, the objects of the disclosure are solved by the steps of: Preferably inserting or connecting a single-use item in/to a medical device; identifying an identifying feature of a connected or inserted single-use item by a detection system, in particular detecting and hence identifying the single-use item and/or identifying a position of the single-use item; preferably, when an identified position of the single-use item does not correspond to the intended position of the single-use item, prohibiting a function of the medical device; filtering and/or limiting, on the basis of the identified single-use item, a drug selection and/or a single-use item selection and/or outputting an alarm and/or prohibiting a therapy with a predefined combination of drug or drug concentration and single-use item.

With respect to a use, the objects are fulfilled in that a single-use item according to the present disclosure is used in a medical device with a detection system and that filtering or limitation of a selection possibility is performed on the basis of the detected identifying feature.

With respect to a computer-readable storage medium the objects are fulfilled in that it comprises instructions which, when executed by a computer, cause same to perform the method steps in accordance with the present disclosure.

It is to be understood that the foregoing disclosures concerning single-use item and single-use item set will also apply as a disclosure for the medical device and the medical system in accordance with the present disclosure and vice versa. It is to be understood that the presently disclosed features of single-use item, single-use item set, medical device, medical system, and medical method are interchangeable and may be supplemented.

### BRIEF DESCRIPTION OF THE DRAWINGS

The present disclosure will be explained in the following by means of preferred embodiment with the aid of Figures.

FIG. 1 is a schematic view of a single-use item in the form of an infusion tube with a clamp in accordance with a preferred embodiment of the present disclosure;

FIG. 2 is a schematic view of a set of single-use items in accordance with a preferred embodiment;

FIG. 3 is a further preferred embodiment of a set of single-use items in accordance with a further preferred embodiment;

FIG. 4 is a schematic view of a medical device in the form of a dialysis machine with an inserted dialysis set as a single-use item set in accordance with a preferred embodiment; and

FIG. 5 is a flowchart of a medical method in accordance with a preferred embodiment.

The Figures are of schematic nature and shall only serve the understanding of the invention. Equal elements are provided with the same reference signs. The features of the different embodiments are interchangeable.

### DETAILED DESCRIPTION

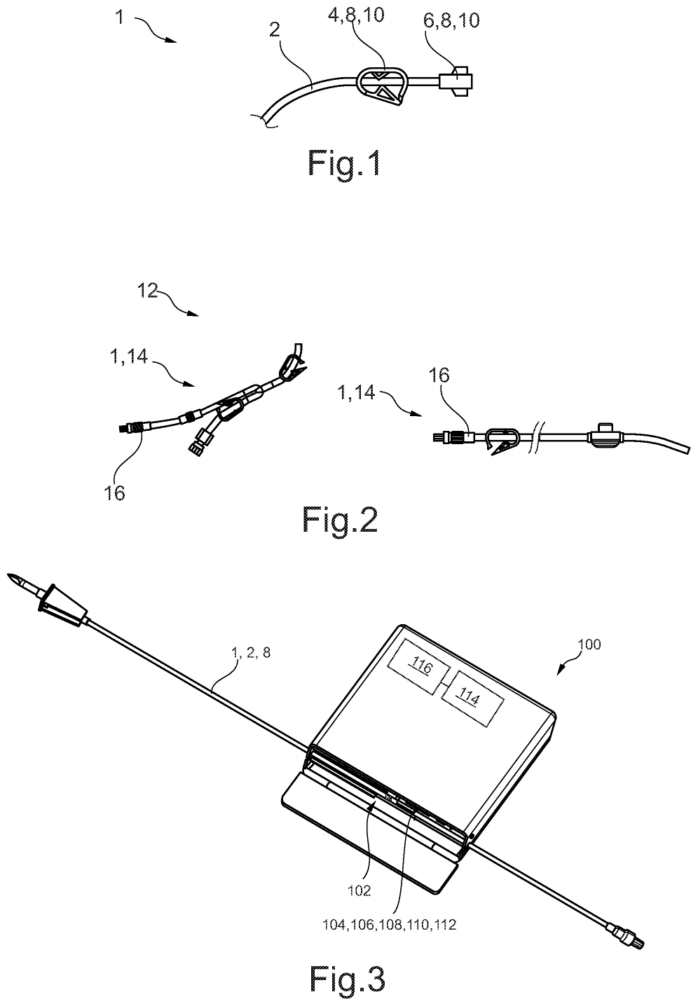

FIG. 1 shows in a perspective, schematic view a single-use item 1 in accordance with a preferred embodiment of the present disclosure. The single-use item 1 is configured in the form of an infusion tube 2 and corpuses an infusion clamp 4 as a further single-use item 1. It can also be said that a single-use item 1 with a further single-use item 1 or two single-use items 1 in accordance with the present disclosure are shown in FIG. 1.

The infusion tube 2 comprises at an end thereof a (terminal) connection 6, for instance, a Luer-Lock connection, which is adapted to be connected to an appropriate counter connection of a medical device. In this embodiment the connection 6 is, as a predefined portion of the infusion tube 2, configured on the whole and plainly in the color green, which acts as a first integrated marker 8 and hence as a first identifying feature 10 of the infusion tube 2. Furthermore, the connection is manufactured of a plastic in which magnetic particles were inserted during manufacturing, and hence comprises magnetisable (magnetic) particles and thus has an own defined and predefined static magnetisation. This magnetisation serves as a second integrated marker 8 of the infusion tube 2, which is integrated therein and is hence inseparably connected therewith.

The two predefined different types or kinds of markers 8 which are inserted at the end portion are suited to be detected visually, on the one hand, and non-visually, on the other hand, via appropriate magnetic sensors. While the signal color green as a first marker 8 visually signalises to a user that this infusion tube 2 is suited for drug administration and can moreover also be detected by means of an appropriate color sensor for implementing automatic examination and identification, the second magnetic marker 8 as a second magnetic identifying feature 10 is only detectable by an appropriate sensor when connected to a medical device. The second magnetic marker 8 serves especially a branding of the infusion tube 2 so as to characterise it as an original part. Moreover, by means of the second magnetic marker 8 as a second identifying feature 10, when connected to a 20) medical device, it is possible to detect an identification of the single-use item 1 as a specific infusion tube 2 by means of a strength of the magnetic field for a drug administration. Also, a correct connection of the infusion tube 2 may be ensured by means of the inherent magnetic field. A magnetic marker with single-use items is completely novel.

In addition, the infusion tube 2 also comprises the attachable infusion clamp 4 which is slidable along the infusion tube 2 and stops a fluid flow by clamping where required. The infusion clamp 4 has a specific conductive polymer as a material so as, in contrast to the state of the art, not to have an electrically insulating configuration, but to comprise a particular conductivity in correspondence with the specific material. In the present case, the infusion clamp 4 has an electric conductivity between 10 and 100 S/m. The (slightly) electrically conductive material of the infusion clamp 4 forms a further type of a marker 8 of the infusion clamp 4. Inserted in a medical device, it is adapted to examine the electric conductivity via two contact connections and to determine that it is the matter of an original product, on the one hand, and, on the other hand, by means of a specific setting of a conductivity, for instance, 10-110 S/m for a drug administration and 1,000 to 10,000 S/m for an enteral solution, an identification and differentiation of two different infusion clamps 4 of an infusion set can take place.

FIG. 2 shows a single-use item set 12 in the form of a dialysis set with two dialysis tube branchings 14 as a single-use item 1. In this embodiment a respective dialysis connection 16 is specifically formed in the first dialysis tube branching 14 as a first single-use item and the second dialysis tube branching 14 as a second single-use item. The first dialysis connection 16 comprises, as a first integrated marker, a fluorescence of a specific wavelength as a color and, as a second integrated marker, a magnetic marker with two small integrated magnets, one at the front side of the dialysis connection 16 and one at the end of the dialysis connection 16. The second dialysis connection 16 also comprises a first integrated marker in the form of the same fluorescence as the first dialysis connection 16, but as a second marker only one magnet at the end of the second dialysis connection 16. By means of the fluorescence it is possible for a medical device with an appropriate fluorescence detection system to examine that it is the matter of an original part or that the single-use item 1 is connected, while the second magnetic marker which is of different configuration in the first and second dialysis connections 16 may be detected via an appropriate magnetic sensor of a detection system and, by means of a stored assignment table of magnetic marker to dialysis tube branchings 14 (single-use item 1), at least one kind of dialysis tube branching 14, in particular exactly one dialysis tube branching 14, can be identified by an appropriately adapted control unit.

FIG. 3 shows a schematic perspective view of a medical system 1000 with a medical device 100 in the form of an infusion pump in accordance with a preferred embodiment. The device 100 comprises a receptacle 102 for an insertable single-use item/accessory item 1 of the infusion pump in the form of a single-use infusion item, here an infusion tube 2 of an infusion bag with an infusion solution. In this embodiment, a UV marker in the form of UV absorbing particles and UV radiation reflecting in the visible range of the light (excitable radiation; inserted during the manufacturing of the infusion tube 2) which are invisible to the naked eye is inserted in the entire infusion tube (in a plastic material). Furthermore, the device 100 comprises a detection system 104 with a UV light 106 and a UV light sensor 108 for detecting an identifying feature adapted to the detection system, here the information in the form of the UV spectrum, of the inserted single-use item 1. Moreover, the detection system 104 comprises an IR light and an IR sensor. In a drug library 114 in a storage unit of the device 100 data concerning drugs are stored. The device 100 comprises additionally a single-use item library in the storage unit (not illustrated here) in which data concerning at least two different infusion tubes 2 as single-use items 1 are stored. In this single-use item library the specific identifying features of the single-use items are stored, in the present case the specific reflection spectrum of the single-use item 1. Concretely, data concerning infusion tubes with IR markers and infusion tubes with UV markers are stored in the single-use item library. IR markers are, for instance, used in infusion tubes 2 by means of which a drug administration is conducted. UV markers are, for instance, used in infusion tubes by means of which an enteral nutrition solution can be supplied. By means of the UV or IR markers of an infusion tube set as a single-use item set 12 with infusion tubes 2 with UV markers, on the one hand, and IR markers, on the other hand, it is then possible to identify a corresponding single-use item and to limit a selection possibility. For instance, when inserting an infusion tube 2 with UV markers, only a selection of an enteral nutrition solution can be indicated to the user as an input possibility. On the other hand, when inserting an infusion tube 2 with IR markers, only a selection possibility of drugs can be indicated to the user.

For this purpose, the device 100 comprises a control unit 116 which is provided and adapted to filter and to limit a drug selection possibility in the drug library 114 by means of the detected infusion tube 2 as a single-use item 1 and to filter and to limit a single-use item selection. Additionally, if a therapy of the medical device 100 with a predefined combination of drug or drug concentration and single-use item is set which is critical, for instance, an incompatible combination of single-use item 1 and selected drug, the control unit 116 may output an alarm via a touch display 118 and/or prohibit start of the therapy. The touch display 118 serves the outputting of information to a user and as an input device for the input of operation input by the user.

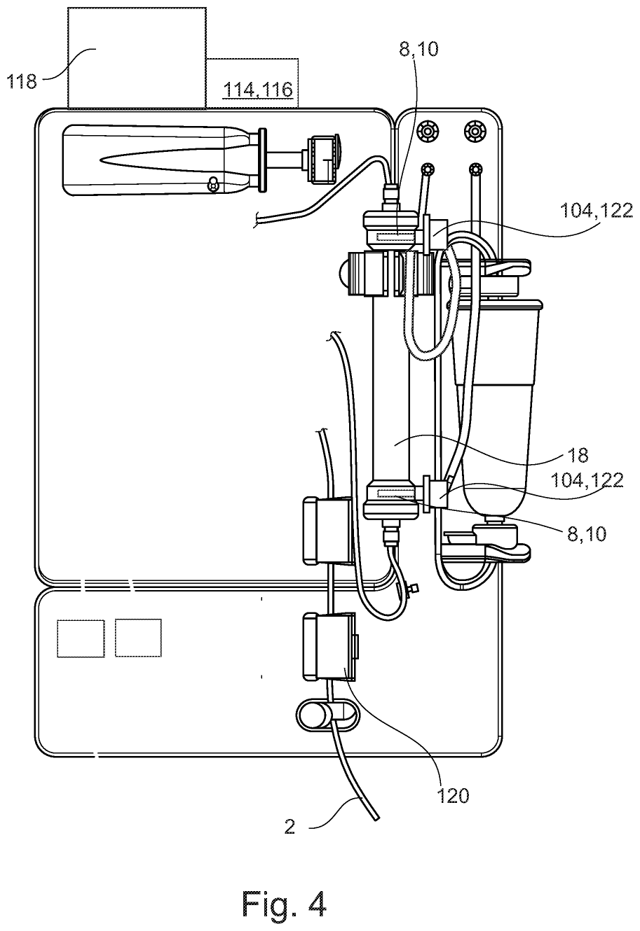

FIG. 4 shows a further preferred embodiment of a medical system 1000 with a medical device 100 in the form of a dialysis machine and an inserted dialysis tube set as a single-use item set 12. Specifically, the dialysis machine comprises an inserted dialysis filter 18 as a further single-use item 1. The dialysis machine comprises, at an upper portion (upper side) and a lower portion (lower side) of the inserted dialysis filter 18, a magnetic sensor 122 so as to detect a magnetic field. The dialysis filter 18 in turn has inserted a magnet as an integrated marker 8 only at an upper side so as to implement a specific marking of the dialysis filter 18. Altogether, at least three different kinds of a dialysis filter can thus be determined (magnet at the top yes, at the bottom no; magnet at the top yes, at the bottom yes; magnet at the top no, at the bottom no). Furthermore, a further differentiation may be made by means of a strength of the magnetisation (magnetic field strength) or an orientation of the magnetic field. The control unit 116 then determines by means of a stored single-use item library which dialysis filter 18 is inserted and automatically sets the parameters for a therapy on the basis of the identified dialysis filter. With respect to the dialysis tube set it comprises, as an integrated marker, a specific portion of the tube, which is usually clamped and which is in contact with a conductivity sensor 120 and has an electric conductivity of 10 to 100 S/m.

The control unit 116 may then perform an appropriate filtering of a dialysis therapy by means of the identified dialysis tube set and may automatically set further parameters. Thus, unintended user errors are excluded from the outset.

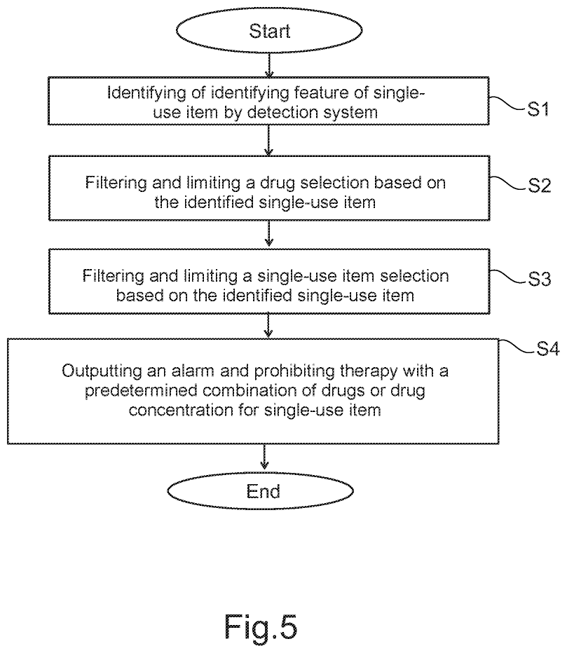

FIG. 5 shows in a flowchart a medical method, in particular an operating method of a preferred embodiment.

In a first step S1 a predefined identifying feature of a single-use item (accessory element) is detected and identified by a detection system.

In a step S2, on the basis of the identified single-use item, a drug selection is filtered and limited, so that the user can only select the drugs matching with the single-use item.

In a step S3, also on the basis of the identified single-use item, a single-use item selection is filtered and limited, so that, for instance, a selection of further single-use items to be inserted is limited.

In a step S4, if a particular critical combination of drug and single-use item is selected or a critical combination of drug concentration and single-use item is selected, an alarm is output and a start of a therapy is prohibited.

1. A single-use medical item for use on or in a medical device, the single-use medical item comprising at least one integrated marker as an identifying feature, wherein the at least one integrated marker is a predefined portion of the single-use medical item having at least one of:
a color;
magnetization;
a geometric shape;
a predefined conductivity; and
an RFID tag embedded in the single-use item.

2. The single-use medical item according to claim 1, wherein the at least one integrated marker comprises at least two markers of a different kind, the at least two markers comprising a first marker as a first identifying feature and a second marker as a second identifying feature predefined portion having a colour, so as to have a combined or two-stage identification performed by the medical device.

3. The single-use medical item according to claim 1, wherein the at least one integrated marker comprises at least two markers of a different kind, the at least two markers comprising a first marker as a first identifying feature comprising a radio transponder and a second marker as a second identifying feature is a comprising the predefined portion having the color.

4. The single-use medical item according to claim 1, wherein the single-use medical item is an infusion tube comprising, at least at a portion, a color coating or an insertion of color pigments.

5. A single-use medical item set comprising:
at least two single-use medical items according to claim 2,
the at least two single-use medical items comprising a first single-use medical item and a second single-use medical item,
the first marker of the first single-use medical item is of comprising a first configuration and the second marker of the second single-use medical item comprising a second configuration different from the first configuration so as to differentiate the first single-use medical item from the second single-use medical item.

6. The single-use medical item set according to claim 5, wherein the first marker of the first single-use medical item is a first magnetization and the first marker of the second single-use medical item is a second magnetization, and
wherein the first magnetization has a first spatial position and a first strength of magnetization, and the second magnetization has a second spatial position and a second strength of magnetization, and
wherein the first spatial position is different from the second spatial position, and/or the first strength of magnetization is different from the second strength of magnetization, so as to differentiate the first single-use medical item from the second single-use medical item.

7. The single-use medical item set according to claim 5,
wherein the first single-use medical item is a first infusion tube and the second single-use medical item is a second infusion tube, and
wherein a first end portion of the first infusion tube comprises a first radially outer surface of a first color, and a second end portion of the second infusion tube comprises a second radially outer surface of a second color different from the first color.

8. A medical device comprising:
a receptacle or a connection for a single-use medical item to be inserted in or connected to the medical device;
a detection system for detecting at least one integrated marker adapted to the detection system as an identifying feature of the single-use medical item; and
a control unit adapted to identify the single-use medical item based on the identifying feature and/or to determine a position of the single-use medical item relative to the detection system.

9. The medical device according to claim 8, further comprising:
a storage unit with a drug library in which data concerning drugs are stored,
wherein the control unit is adapted to:
filter and/or limit a drug selection possibility in the in the drug library,
filter and/or limit a selection possibility of a further single-use medical item, and/or
output an alarm and/or prohibit a start of a therapy of the medical device when setting the therapy with a predefined combination of a drug or drug concentration and a single-use medical item.

10. The medical device according to claim 8, wherein the detection system comprises a magnetic sensor configured to detect a magnetic field of the single-use medical item as an identifying feature, and a color sensor configured to detect a color of a predefined portion of the single-use medical item received or connected, wherein the control unit is configured, in a first stage, to determine a correct position and a correct reception or a correct connection of the single-use medical item based on the magnetic field and to identify the single-use medical item based on the color in a second stage when a correct position of the single-use item is detected.

11. The medical device according to claim 8, further comprising a position determining module with a stored assignment of communications network to position determination,
wherein the control unit is configured to, based on a determined position of the medical device, configured:
filter and/or limit a drug selection possibility in the drug library, and/or
filter and/or limit a selection possibility of the single-use medical item.

12. A medical system, comprising:
a medical device according to claim 9; and
a single-use medical item set comprising:
a first single-use medical item and a second single-use medical item,
the first single-use medical item comprising a first integrated marker of a first configuration, and the second single-use medical item comprising a second integrated marker of a second configuration different from the first configuration,
the first integrated marker and the second integrated marker each being a predefined identifying feature that is detectable by the detection system of the medical device,
the second configuration being different from the first configuration so as to differentiate the first single-use medical item from the second single-use medical item.

13. The medical system according to claim 12, wherein:
the first integrated marker and the second integrated marker each comprise an RFID tag,
the medical device comprises an RFID reader digitally providing read-out data of each RFID tag to the control unit, and
the control unit is configured to limit, based on the patient library with data stored for patients, the drug selection possibility in the drug library and to display the drug selection possibility via a display to a user.

14. A method of operating the medical device according to claim 10, the medical method comprising the steps of:
identifying the identifying feature of the single-use medical item by the detection system and thus identifying the single-use medical item and/or a position of the single-use medical item; and
filtering and/or limiting, based on the single-use medical item, a drug selection and/or a single-use item selection and/or outputting an alarm and/or prohibiting a therapy.

15. A computer-readable storage medium comprising instructions which, when executed by a computer, cause the computer to perform the method of operating the medical device according to claim 14.
